(19) United States
(12) Patent Application Publication
(10) Pub. No.: US 2005/0089518 A1
(43) Pub. Date: Apr. 28, 2005

(54) Prospective identification and characterization of breast cancer stem cells

(76) Inventors: Michael F. Clarke, Ann Arbor, MI (US); Max S Wicha, Ann Arbor, MI (US); Muhammad Al-Hajj, Cambridge, MA (US)

Correspondence Address: David A Casimir;Medlen & Carroll, Suite 350, 101 Howard Street, San Francisco, CA, 94105, US

(21) Appl. No.: 10/497,791

(22) PCT Filed: Dec. 6, 2002
(86) PCT No.: PCT/US02/39191

Related U.S. Application Data
(60) Provisional application No. 60/338,358, filed on Dec. 7, 2001.

(51) Int. Cl. A61K 39/395; A61K 38/17

(52) U.S. Cl. 424/143.1; 514/2

(57) ABSTRACT

Human breast tumors contain hetrogeneous cancer cells. using an animal xenograft model in which human breast cancer cells were grown in immunocompromised mice we found that only a small minority of breast cancer cells had capacity to form new tumors. The ability to form new tumors was not a slochastic property, rather certain populations of cancer cells were depleted for the ability to form new tumors, while other populations were enriched for the ability to form new tumors. Tumorigenic cells could be distinguished from non-tumorigenic cancer cells based on surface marker expression. We prospectively identified and isolated the tumorigenic cells as CD44³⁰CD24^{-/low}LINEAGE A few as 100 cells from this population were able to form tumors the animal xenograft model, while tens of thousands of cells from non-tumorigenic populations failed to form tumors. The tumorigenic cells could be serially passaged, each time generating new tumors containing and expanded numbers of CD44^{+CD24} Lineage tumorigenic cells as well as phenotypically mixed populations of non-tumorigenic cancer cells. This is reminiscent of the ability of normal stem cells to self-renew and differentiate. The expression of potential therapeutic targets also differed between the tumorigenic and non-tumorigenic populations. Notch activation promoted the survival of the tumorigenic cells, and a blocking antibody against Notch 4 induced tumorigenic breast cancer cells to undergo apoptosis.

### TECHNICAL FIELD OF THE INVENTION

This invention relates general to the investigation or analysis of biological materials by determining their chemical or physical properties, and in particular to the diagnosis and treatment of cancer.

### BACKGROUND ART

Breast cancer is the most common cancer in women, but metastatic breast cancer is still incurable. Despite advances in detection and treatment of metastatic breast cancer, mortality from this disease remains high because current therapies are limited by the emergence of therapy-resistant cancer cells. As a result, metastatic breast cancer remains an incurable disease using current treatment strategies.

In solid tumors generally, only a small proportion of the tumor cells are able to form colonies in an in vitro clonogenic assay. Large numbers of cells must typically be transplanted to form tumors in vivo. These observations have been explained by a stochastic model in which each tumor cell has the capacity to proliferate and form new tumors but only a small proportion of the cells is able to exhibit this capacity at any one time.

Alternatively, only a rare subset of solid tumor cells may have the capacity to significantly proliferate or form new tumors, but cells within this subset may do so very efficiently. If only a small, identifiable subset of solid tumors cells possesses the capacity to proliferate and form new solid tumors, this would have important implications for cancer therapy. To eradicate solid tumors, it would be necessary to kill this subpopulation of cells.

The prospective identification and isolation of hematopoietic stem cells and nervous system stem cells has brought about rapid advances in our understanding of these cells. Thus, if it is possible to prospectively identify and isolate a tumorigenic cell population, it would then be possible to much more effectively focus the development anti-solid tumor therapeutics and diagnostics.

### DISCLOSURE OF THE INVENTION

The invention is based upon the discovery that a small percentage of tumorigenic cells within an established solid tumor have the properties of stem cells. These solid tumor stem cells give rise both to more solid tumor stem cells and to the majority of cells in the tumor, cancer cells that have lost the capacity for extensive proliferation and the ability to give rise to new tumors. Thus, solid tumor cell heterogeneity reflects the presence of a variety of tumor cell types that arise from a solid tumor stem cell.

This invention provides a way that anti-cancer therapies can be directed, both generally and now specifically directed, against the solid tumor stem cells. The previous failure of cancer therapies to significantly improve outcome has been due in part to the failure of these therapies to target the solid tumor stem cells within a solid tumor that have the capacity for extensive proliferation and the ability to give rise to all other solid tumor cell types. Effective treatment of solid tumors thus requires therapeutic strategies that are able to target and eliminate the tumorigenic subset of solid tumor cells, i.e., the solid tumor stem cells, by the direct targeting of therapeutics to the solid tumor stem cells. Accordingly, the invention provides a method for reducing the size of a solid tumor, by contacting the cells of the solid tumor with a therapeutically effective amount of an agent directed against a Notch4 polypeptide. Inhibition of Notch4-signaling impairs the growth of the solid tumor stem cells. The invention also provides a method for reducing the size of a solid tumor; by contacting the cells of the solid tumor with a therapeutically effective amount of an agent that modulates the activity of Maniac Fringe.

The invention provides in vivo and in vitro assays of solid tumor stem cell function and cell function by the various populations of cells isolated from a solid tumor. The invention provides methods for using the various populations of cells isolated from a solid tumor (such as a population of cells enriched for solid tumor stem cells) to identify factors influencing solid tumor stem cell proliferation. By the methods of the invention, one can characterize the phenotypically heterogeneous populations of cells within a solid tumor. In particular, one can identify, isolate, and characterize a phenotypically distinct cell population within a tumor having the stem cell properties of extensive proliferation and the ability to give rise to all other tumor cell types. Solid tumor stem cells are the truly tumorigenic cells that are capable of re-establishing a tumor following treatment.

The invention thus provides a method for selectively targeting diagnostic or therapeutic agents to solid tumor stem cells. The invention also provides an agent, such as a biomolecule, that is selectively targeted to solid tumor stem cells.

In its several aspects, the invention usefully provides methods for screening for anti-cancer agents; for the testing of anti-cancer therapies; for the development of drugs targeting novel pathways; for the identification of new anti-cancer therapeutic targets; the identification and diagnosis of malignant cells in pathology specimens; for the testing and assaying of solid tumor stem cell drug sensitivity; for the measurement of specific factors that predict drug sensitivity; and for the screening of patients (e.g., as an adjunct for mammography).

### BRIEF DESCRIPTION OF DRAWINGS

FIG. 1 shows the isolation of tumorigenic cells. Flow cytometry was used to isolate subpopulations of Tumor 1 (T1; FIG. 1a, FIG. 1b), Tumor 3 (T2; FIG. 1c), Tumor 5 (T5; FIG. 1d), Tumor 6 (T6; FIG. 1e) and Tumor 7 (T7; FIG. 1f) cells, which were tested for tumorigenicity in NOD/SCID mice. T1(FIG. 1b) and T3 (FIG. 1c) had been passaged (P) once in NOD/SCID mice. The rest of the cells were frozen or unfrozen samples obtained directly after removal from a patient (UP). Cells were stained with antibodies against CD44, CD24, LINEAGE markers, and mouse-H2K (for passaged tumors obtained from mice), and 7AAD. Dead cells (7AAD⁺), mouse cells (H2K⁺) and LINEAGE⁺ normal cells were eliminated from all analyses. Each plot depicts the CD24 and CD44 staining patterns of live human LINEAGE⁻ cancer cells, and the frequency of the boxed tumorigenic cancer population as a percentage of cancer cells/all cells in each specimen is shown. Tumor 3 (T3) cells were stained with Papanicolaou stain and examined microscopically (100× objective). Both the non-tumorigenic (FIG. 1g) and tumorigenic (FIG. 1h) populations contained cells with a neoplastic appearance, with large nuclei and prominent nucleoli. Histology from the CD24⁺ injection site (FIG. 1i; 20× objective magnification) revealed only normal mouse tissue while the CD24^{-/low} injection site (FIG. 1j; 40× objective magnification) contained malignant cells (FIG. 1k). A representative tumor in a mouse at the CD44⁺CD24^{-/low}LINEAGE³¹ injection site, but not at the CD44⁺CD24⁺ LINEAGE⁻ injection site.

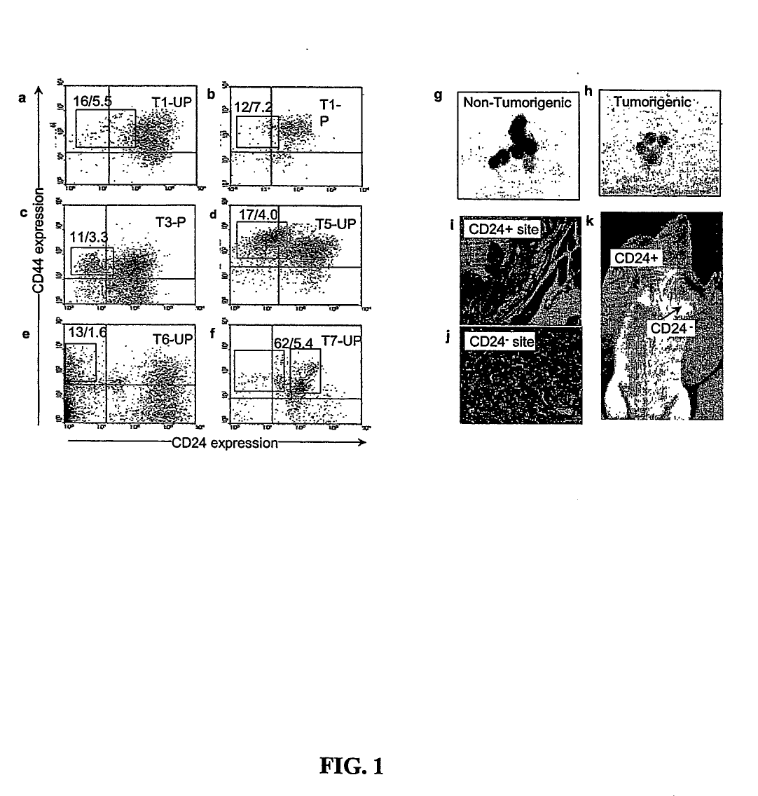

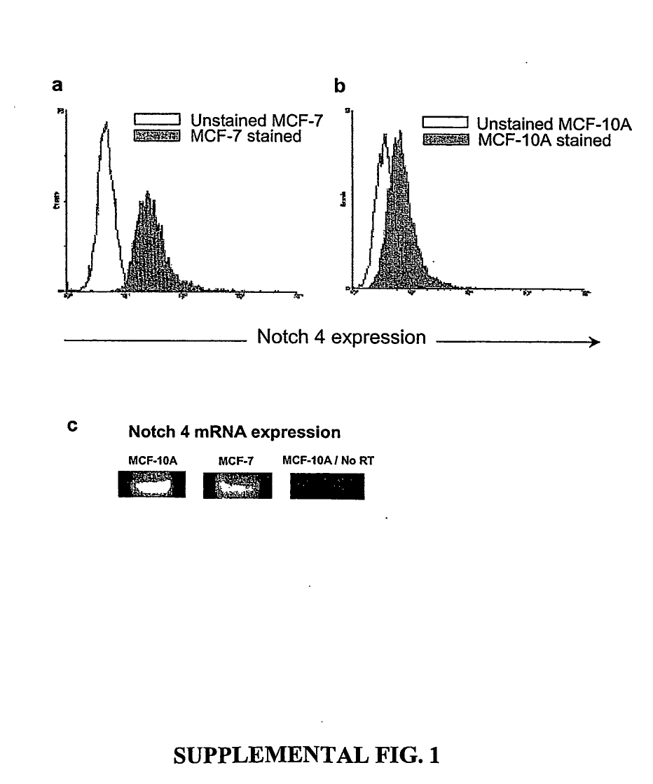

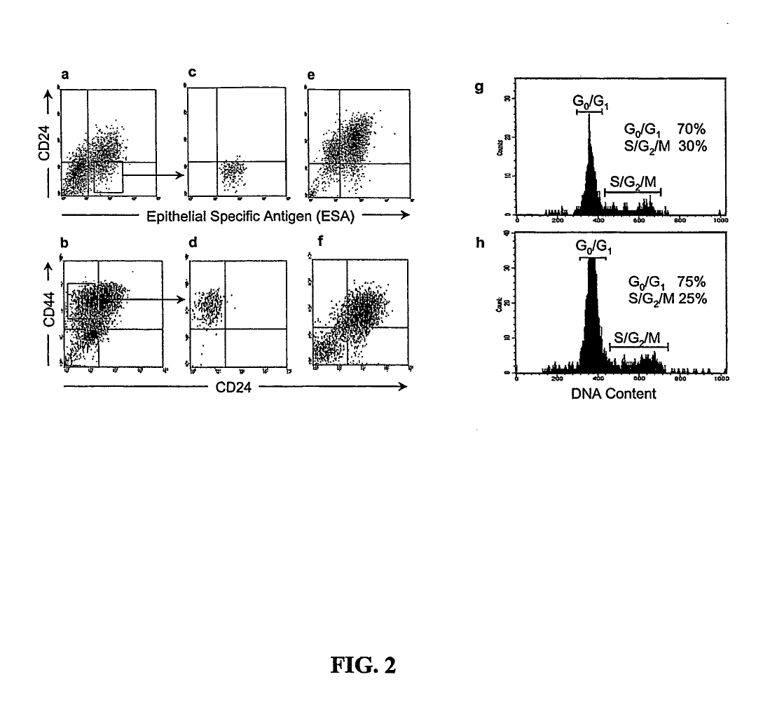

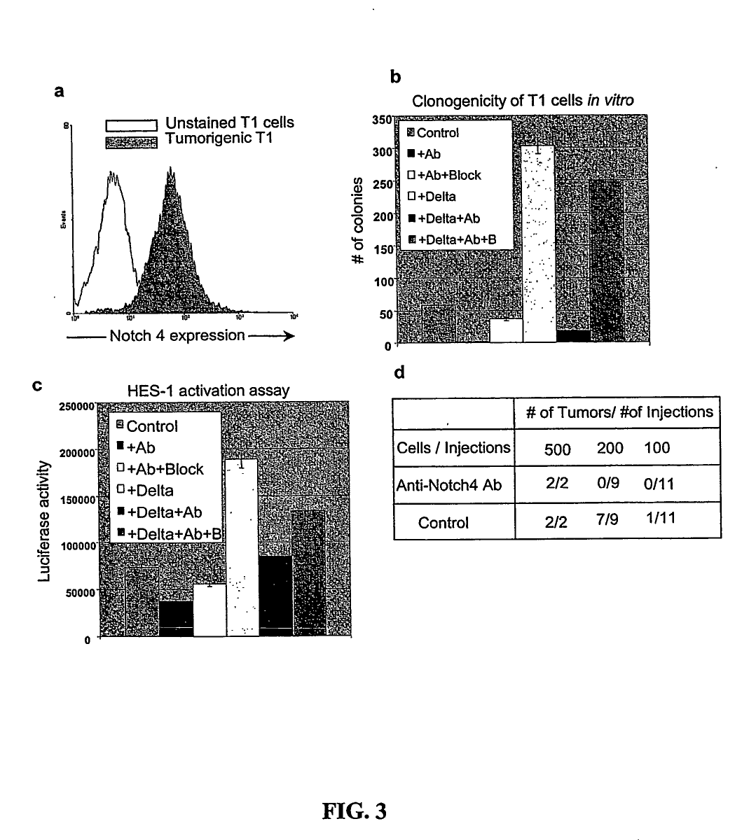

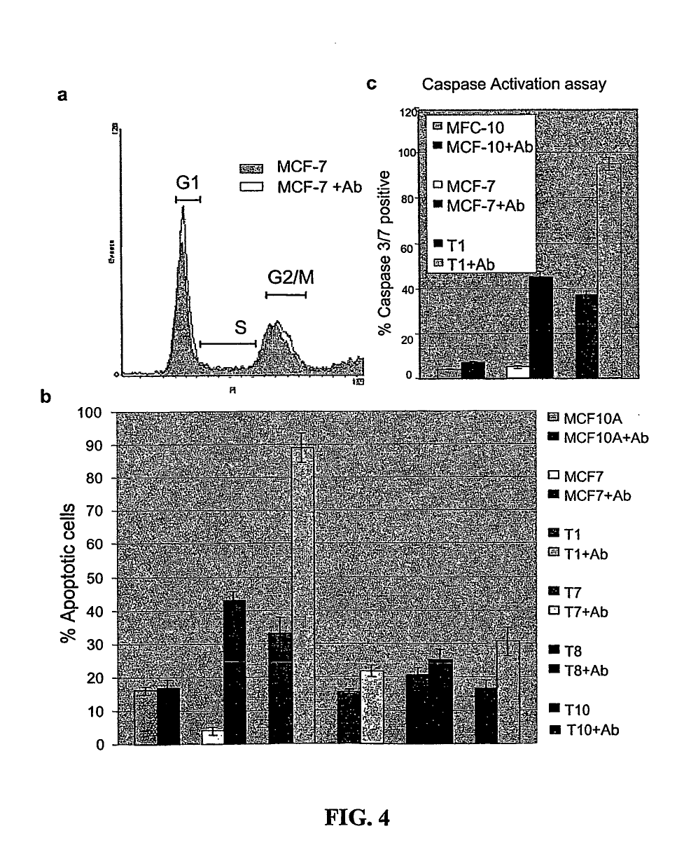

Supplemental FIG. 1 shows the expression of Notch4 by MCF-7 and MCF-10 cells. MCF-7 cells (Supplemental FIG. 1a) and MCF-10 cells (Supplemental FIG. 1b) were stained with the anti-Notch4 antibody. T1 cells and MCF-7 cells express higher levels of the protein than MCF-10 cells. (Supplemental FIG. 1c) RT-PCR was done using nested primers to detect expression of Notch4 MRNA. Notch4 was expressed by MCF-7 cells, and MCF-10 cells. The message was not detected when reverse transcriptase (RT) was omitted from the reaction (MCF10/no RT). We confirmed that the MCF-7 cells expressed Notch4 at both the RNA and protein levels. These data were independently confirmed using two different pairs of intron spanning Notch4-specific PCR primers. Note, it is possible that different sublines of “NCF-7” cells in circulation can differ in their expression of Notch4. Osbome CK et al., Breast Cancer Research & Treatment. 9: 111-121 (1987).

FIG. 2 shows the phenotypic diversity in tumors arising from solid tumor stem cells. The plots depict the CD24 and CD44 or ESA staining patterns of live human LINEAGE⁻ cancer cells from Tumor 1 (T1; FIG. 2a, FIG. 2c and FIG. 2e) or Tumor 2 (T2; FIG; 2b, FIG. 2d and FIG. 2f). T1 CD44⁺LINEAGE⁻ cells (FIG. 2a) or T2 LINEAGE⁻ cells (FIG. 2b) were obtained from tumors that had been passaged once in NOD/SCID mice. ESA⁺CD44⁺CD24^{-/low} LINEAGE⁻ tumorigenic cells from T1(FIG. 2c) or CD44⁺CD24^{-/low} LINEAGE⁻ tumorigenic cells from T2 (FIG. 2d) were isolated and injected into the breasts of NOD/SCID mice. Plots shown in FIG. 2e and FIG. 2f depict analyses of the tumors that arose from these cells. In both cases, the tumorigenic cells formed tumors that contained phenotypically diverse cells similar to those observed in the original tumor. The cell cycle status of the ESA⁺CD44⁺CD24^{-/low} LINEAGE⁻ tumorigenic cells (FIG. 2g) and the remaining LINEAGE⁻ non-tumorigenic cancer cells (FIG. 2h) isolated from T1were determined by hoechst 33342 staining of DNA content, according to the method of Morrison SJ & Weissman IL, Immunity 1: 661-673 (1994). The tumorigenic and non-tumorigenic cell populations exhibited similar cell cycle distributions.

FIG. 3 shows that blocking antibodies against Notch4 inhibited colony formation by solid tumor stem cells. FIG. 3a shows Notch4 expression by T1tumorigenic breast cancer cells. Tumorigenic (CD44⁺CD24^{-/low} LINEAGE³¹ ) T1cells that had been passaged once in NOD/SCID mice were stained with the anti-Notch4 antibody. FIG. 3b shows colony formation/unsorted 20,000 T1 cells grown for 14 days in the indicated tissue culture medium supplemented with Fc antibody (control); polyclonal anti-Notch4 antibody (Ab); polyclonal anti-Notch4 antibody plus blocking peptide (Ab+Block); Delta-Fc (Delta); Delta plus anti-Notch4 Ab (Delta+Ab); and Delta plus polyclonal anti-Notch4 antibody plus blocking peptide (Delta+Ab+B). Soluble Delta-Fc (Delta) stimulated colony formation (p<0.001), while the polyclonal anti-Notch4 antibody (Ab) inhibited colony formation in the presence of Delta-Fc (Delta+Ab) (p<0.001). When the antibody was pre-incubated with the peptide used to generate the anti-Notch4 antibody, the inhibitory effect of the antibody was nearly completely reversed (Ab+Block; Delta+Ab+Block; p<0.001). FIG. 3b is a Notch pathway reporter gene assay showing that soluble delta-Fc (Delta) activated Notch relative to control Fc construct (Control). Anti-Notch4 polyclonal antibody (Ab) inhibited Notch activation, even in the presence soluble Delta-Fc (Delta+Ab). Addition of a blocking peptide against which the polyclonal antibody was made (Block) partially reversed the ability of the antibody to inhibit Notch activation (Delta+Ab+Block). In FIG. 3d, ESA⁺CD44⁺CD24^{-/low} LINEAGE⁻ tumorigenic cells were isolated from first or second passage T1tumor. The indicated number of cells were injected into the area of the mammary fat pads of mice in control buffer or after being. incubated with a polyclonal anti-Notch4 antibody. Tumor formation was monitored over a five-month period. Note that tumor formation by 500 antibody-treated cells was delayed by an average of three weeks.

FIG. 4 shows that Notch4 signaling provides a survival signal to tumor-initiating cells. FIG. 4a shows the cell cycle status of control MCF-7 cells (shaded) and MCF-7 cells 24 hrs after exposure to the anti-Notch4 antibody (open) was determined by PI staining of DNA content according to the methods of Clarke M F et al., Proc. Natl. Acad. Sci. USA 92: 11024-11028 (1995) and Ryan J J et al., Mol. & Cell. Biol. 1: 711-719 (1993). Each group exhibited similar cell cycle distributions. FIG. 4b shows PI³⁰ apoptotic MCF-10, MCF-7, ESA⁺CD44⁺CD24^{-/low}LINEAGE⁻ tumorigenic Tumor 1 (T1) cells grown in media for 48 hours, or H2K⁻ Tumor 7 (T7), Tumor 8 (T8), or Tumor 10 (T10) cells grown in media for 5 days with (+Ab) or without the anti-Notch4 antibody were identified by flow cytometry. The timing of the onset of apoptosis after antibody addition was similar to that seen after some other death signals. Clarke M F et al., Proc. Natl. Acad. Sci. USA 92: 11024-11028 (1995)(bcl-xs); Ryan J J et al., MoL & Cell. BioL 1: 711-719 (1993) (p53)). Note that the antibody was lethal to the T1and T10 cells. FIG. 4c shows that at forty-eight hours after exposure to the anti-Notch4 antibody, the percentage of cells expressing activated caspase 3 and/or 7, as measured by flow cytometry using the fluorogenic substrate CaspoTag™, was markedly increased in T1tumor-initiating cells and MCF-7 cells, but not MCF-10 cells, as compared to control cells. Tumor 1 (T1) tumorigenic (ESA⁺CD44⁺CD24^{-/low}LINEAGE⁻) cells were isolated by flow cytometry as described in TABLE 3.

### MODES FOR CARRYING OUT THE INVENTION

Introduction. By this invention, the principles of normal stem cell biology have been applied to isolate and characterize solid tumor stem cells generally. Solid tumor stem cells are defined structurally and functionally as described herein; using the methods and assays similar to those described below. Solid tumor stem cells undergo “self-renewal” and “differentiation” in a chaotic development to form a tumor, give rise to abnormal cell types, and may change over time as additional mutations occur. The functional features of a solid tumor stem cell are that they are tumorigenic, they give rise to additional tumorigenic cells (“self-renew”), and they can give rise to non-tumorigenic tumor cells (“differentiation”). The developmental origin of solid tumor stem cells can vary between different types of solid tumor cancers. Typically, solid tumors are visualized and initially identified according to their locations, not by their developmental origin. Accordingly, one can use the method of the invention, employing the markers disclosed herein, which are consistently useful in the isolation or identification of solid tumor stem cells in a majority of patients.

Examples of solid tumors from which solid tumor stem cells can be isolated or enriched for according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. The invention is particularly applicable to sarcomas and epithelial cancers, such as ovarian cancers and breast cancers.

“Enriched”, as in an enriched population of cells, can be defined based upon the increased number of cells having a particular marker in a fractionated set of cells as compared with the number of cells having the marker in the unfractionated set of cells. However, the term “enriched” can be preferably defined by tumorigenic function as the minimum number of cells that form tumors at limit dilution frequency in test mice. The solid tumor stem cell model provides the linkage between these two definitions of (phenotypic and functional) enrichment.

In particular, we have found that breast cancers contain heterogeneous populations of neoplastic cells. Using a xenograft model in which human breast cancer cells were grown in immunocompromised mice, we found that only a small minority of breast cancer cells had the capacity to form new tumors. The ability to form new tumors was not a stochastic property. Rather, certain populations of cancer cells were depleted for the ability to form new tumors while other populations were enriched for the ability to form new tumors. Indeed, we could consistently predict which cells would be most tumorigenic based on surface marker expression.

Using the methods of the invention, we prospectively identified and isolated the tumorigenic cells as CD44⁺CD24^{-/low}LINEAGE⁻. As few as 100 cells from this population were able to form tumors in immunocompromised mice, while tens of thousands of cells from non-tumorigenic populations failed to form tumors. The CD44⁺CD24^{-/low}LINEAGE⁻ cells displayed stem cell-like properties in that they were capable of generating new tumors containing additional CD44⁺CD24^{-/lo}LINEAGE⁻ tumorigenic cells as well as the phenotypically mixed populations of non-tumorigenic cells present in the original tumor. The expression of potential therapeutic targets also differed between the tumorigenic and non-tumorigenic populations of cancer.

Inhibition of Notch4-signaling impaired the growth of the breast cancer stem cells in vitro and in vivo. Effective treatment of solid tumors thus requires therapeutic strategies that are able to target and eliminate the tumorigenic subset of solid tumor cells, i.e., the solid tumor stem cells, by the direct targeting of therapeutics to the solid tumor stem cells.

Animal xenograft model. To test whether solid cancer cells vary in their potential to form new tumors according to the predictions of cancer cell heterogeneity models, we developed an animal xenograft model in which primary or metastatic human breast cancers could efficiently and reproducibly be grown and analyzed in immunodeficient mice. We used a modification of the NOD/SCID immunodeficient mouse model, in which human breast cancers were efficiently propagated in the area of the mouse mammary fat pad. See, Sakakibara T et al., Cancer J. Sci. Am. 2: 291-300 (1996). See also, published PCT patent application WO 02/12447, the entire contents of which are incorporated herein by reference.

Thus, the invention provides an animal xenograft model in which to establish tumors by the injection of solid tumor cells into a host animal. The host animal can be a model organism such as nematode, fruit fly, zebrafish; preferably a laboratory mammal such as a mouse (nude mouse, SCID mouse, NOD/SCID mouse, Beige/SCID Mouse), rat, rabbit, or primate. The severely immunodeficient NOD-SCID mice were chosen as recipients to maximize the participation of injected cells. Immunodeficient mice do not reject human tissues, and SCID and NOD-SCID mice have been used as hosts for in vivo studies of human hematopoiesis and tissue engraftinent. McCune et al., Science 241: 1632-9 (1988); Kamel-Reid & Dick, Science 242: 1706-9 (1988); Larochelle et al., Nat. Med. 2: 1329-37 (1996). In addition, Beige/SCID mice also have been used. NOD/SCID mice have previously been validated as in vivo models for the growth of normal human hematopoietic stem cells (Larochelle A et al., Nature Medicine 2: 1329-1337 (1996); Peled A et al., Science 283: 845-8 (1999); Lapidot T et al., Science 255: 1137-1141 (1992)) human neural stem cells (Uchida N et al., Proc. Natl. Acad. Sci. USA 97:14720-5 (2000)) and human acute myelogenous leukemia (AML) stem cells (Lapidot T et al., Nature 17: 367:645-648. (1994); Bonnet D & Dick J E, Nature Medicine 3: 730-737 (1997)). Another useful mouse is the β2 microglobin deficient NOD/SCID mouse. Kollet O et al., Blood 95: 3102-3105 (2000).

Some previous clonogenic in vitro assays of cancer cells were difficult to interpret since cells from different tumors proliferated to different extents and only occasionally yielded cells that could be serially passaged indefinitely (immortal cells). Similarly, some previous in vivo assays of tumorigenicity were difficult to interpret because cancer cells from some patients engrafted while pathologically similar cancer cells from other patients failed to engraft. By contrast, the animal xenograft model of this invention permitted tumor formation by all the patient samples that were tested.

In the assays described below, 8-week old female NOD-SCID mice were anesthetized and then injected IP with etoposide (30 mg/kg). At the same time, estrogen pellets were placed subcutaneously on the back of the neck using a trocar. All tumor injections/implants were performed five days after this procedure. For the implantation of fresh specimens, samples of human breast tumors were received within an hour after the surgeries. The tumors were minced to yield 2-mm³ pieces. A 2-mm incision was then made in the mouse and a 2-mm piece of a primary tumor was inserted or 10⁷ pleural effusion cells were injected into the breast. A 6-0 suture was wrapped twice around the breast and nipple to hold the implanted pieces in place. Nexaban was used to seal the incision and mice were monitored weekly for tumor growth. For the injection of cancer cells from pleural effusions, cells were received shortly after thoracentesis and washed with HBSS. Viable cell numbers were counted during sorting and verified using a hemocytometer. After centrifugation, the indicated number of cells were suspended in 100 μl of a serum free-RPMI/Matrigel® mixture (1:1 volume). A nick was made approximately 1-cm form the nipple, and an 18-gauge needle was inserted and tunneled into the subcutaneous tissue immediately under the nipple. The cells were then injected in the area of the mammary fat pad. The site of the needle injection was sealed with Nexaban to prevent cell leakage.

Other general techniques for formulation and injection of cells into the animal xenograft model may be found in Remington's Pharmaceutical Sciences, 20th ed. (Mack Publishing Co., Easton, Pa.). Suitable routes may include parenteral delivery, including intramuscular, subcutaneous, intramedullary injections, as well as intrathecal, direct intraventricular, intravenous, intraperitoneal, intranasal, or intraocular injections, just to name a few. For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

In the assays discussed below, the animals were injected with unsorted T1 and T3 cells, and a 2-mm piece of T2. Injected ells from T4-T9 were isolated by flow cytometry as described in FIG. 1 and TABLE 3. Solid tumor cells for injection were obtained from a primary breast tumor (T2) as well as from metastatic pleural effusions (T1, T3-T9). Some assays were performed on cells after they had been passaged once in mice (Passage 1; see, TABLE 3 below) while other assays were performed on unpassaged fresh or frozen tumor samples obtained directly from patients (Unpassaged; see, TABLE 3 below). For cell culture, Passage-1 primary breast cancer cells were plated in triplicate 12-well dishes in HAM-F12 medium supplemented with Fetal Bovine Serum (1%), Insulin (5 μg/ml), Hydrocortisone (1 μg/ml), EGF (10 μg/ml), Choleratoxin (0.1 μg/ml), Transfenrin and Selenium (GIBCO BRL, recommended dilutions), pen/strep, and fungizone (Gibco/BRL). Culture medium was replaced once every two days.

As shown in TABLE 1 below, all of the solid tumor specimens that were available to us engrafted in the animal xenograft model. Breast cancer cells were obtained from nine different patients (designated tumors 1-9; T1-T9) and grown in the animal xenograft model model.

TABLE 1
Engraftment of Solid Tumor Cells into the Animal Xenograft Model

| | Tumor | Tumor formation | |
|---|---|---|---|
| Tumor | origin | in mice | Serial passage in mice |
| T1 | Metastasis | Yes | Yes |
| T2 | Breast primary | Yes | Yes |
| T3 | Metastasis | Yes | Yes |
| T4 | Metastasis | Yes | No |
| T5 | Metastasis | Yes | Yes |
| T6 | Metastasis | Yes | Yes |
| T7 | Metastasis | Yes | Yes |
| T8 | Metastasis | Yes | Yes |
| T9 | Metastasis | Yes | Yes |

The tumors passaged in the animals contained heterogeneous cancer cells that were phenotypically similar to the cancer cells present in the original tumors from patients (see, e.g., FIG. 1a and FIG. 1b), including both tumorigenic and non-tumorigenic fractions. This result demonstrates that the environment of the animal xenograft model was not incompatible with the survival of the non-tumorigenic cell fractions. Both the tumorigenic and non-tumorigenic fractions of cancer cells exhibited a similar cell-cycle distribution in mouse tumors (FIG. 2g and FIG. 2h), demonstrating that the non-tumorigenic cells were able to divide in mice.

In summary, we did not encounter a specimen from which a significant number of cancer cells could be recovered that then failed to engraft. Only one sample failed to serially passage in the mice. Thus, the tumors and tumorigenic cells characterized here are representative of all the breast cancer specimens that were available to us, rather than a subset that was selected for the ability to grow in the assay. Moreover, we have used the animal xenograft model to grow sarcoma cells. Thus, the animal xenograft model reliably supports the engraftment of clonogenic human progenitors, i.e., solid tumor stem cells.

Characterization of tumorigenic solid tumor stem cells. As described above, solid tumor stem cells can be operationally characterized by cell surface markers. These cell surface markers can be recognized by reagents that specifically bind to the cell surface markers. For example, proteins, carbohydrates, or lipids on the surfaces of solid tumor stem cells can be immunologically recognized by antibodies specific for the particular protein or carbohydrate (for construction and use of antibodies to markers, see, Harlow, Using Antibodies: A Laboratory Manual (Cold Spring Harbor Press, Cold Spring Harbor, N.Y., 1999)). The set of markers present on the cell surfaces of solid tumor stem cells (the “cancer stem cells” of the invention) and absent from the cell surfaces of these cells is characteristic for solid tumor stem cells. Therefore, solid tumor stem cells can be selected by positive and negative selection of cell surface markers. A reagent that binds to a solid tumor stem cell is a “positive marker” (i.e., a marker present on the cell surfaces of solid tumor stem cells) that can be used for the positive selection of solid tumor stem cells. A reagent that binds to a solid tumor stem cell “negative marker” (i.e., a marker not present on the cell surfaces of solid tumor stem cells but present on the surfaces of other cells obtained from solid tumors) can be used for the elimination of those solid tumor cells in the population that are not solid tumor stem cells (i.e., for the elimination of cells that are not solid tumor stem cells).

The discrimination between cells can be based upon the detected expression of cell surface markers is by comparing the detected expression of the cell surface marker as compared with the mean expression by a control population of cells. For example, the expression of a marker on a solid tumor stem cell can be compared to the mean expression of the marker by the other cells derived from the same tumor as the solid tumor stem cell. Other methods of discriminating among cells by marker expression include methods of gating cells by flow cytometry based upon marker expression (see, Givan A, Flow Cytometry: First Principles, (Wiley-Liss, New York, 1992); Owens M A & Loken M R, Flow Cytometry: Principles for Clinical Laboratory Practice, (Wiley-Liss, New York, 1995)).

A “combination of reagents” is at least two reagents that bind to cell surface markers either present (positive marker) or not present (negative marker) on the surfaces of solid tumor stem cells, or to a combination of positive and negative markers. The use of a combination of antibodies specific for solid tumor stem cell surface markers results in the method of the invention being useful for the isolation or enrichment of solid tumor stem cells from a variety of solid tumors, including sarcomas, ovarian cancers, and breast tumors. Guidance to the use of a combination of reagents can be found in published PCT patent application WO 01/052143, incorporated by reference.

To prepare cells for flow cytometric analysis in the assays described herein, single cell suspensions of solid tumors or pleural effusions were made by mincing solid tumors and digesting them with 200 μ/ml of collagenase 3 (Worthington) in M119 medium (Gibco/BRL, Rockville, Md. USA) for 2-4 hours at 37° C. with constant agitation. Antibodies included anti-CD44, anti-CD24, anti-B38.1, anti-EGFR, anti-HER2/neu, anti-ESA-FITC (Biomeda, Calif. USA), anti-H2K, and goat-anti-human Notch4 (Santa Cruz Products, Santa Cruz, Calif. USA). CD44 (Saddik M & Lai R, J. Clin. Pathol. 52(11): 862-4 (1999)) and CD24 (Aigner S et al., Blood: 89(9): 3385-95 (1997)) are adhesion molecules. B38.1 has been described as a breast/ovarian cancer-specific marker (Ahrens T et al., Oncogene 20: 3399-408, (2001); Uchida N et al., Proc. Natl. Acad. Sci. USA 97: 14720-5 (2000); Kufe D W et al., Cancer Research 43: 851-857 (1983)). LINEAGE marker antibodies were anti-CD2, -CD3-CD10, -CD16, -CD18, -CD31, -CD64 and -CD140b. Unless noted, antibodies are available from Pharmingen (San Diego, Calif. USA). Antibodies were directly conjugated to various fluorochromes depending on the assay. Dissociated tumor cells were stained with anti-CD44, anti-CD24, anti-B38.1, anti-EGFR, anti-HER2/neu, anti-ESA, anti-H2K, Streptavidin-Phar-red, goat-anti-human Notch4, donkey anti-goat Ig-FITC, anti-LINEAGE-Cytochrome (LINEAGE antibodies were anti-CD2, -CD3-CD10, -CD14, -CD18, -CD31, -CD64 and -CD140b) each directly conjugated to a fluor except H2k which was biotinylated. Mouse cells and/or LINEAGE⁺ cells can be eliminated by discarding H2K⁺ (class I MHC) cells or LINEAGE⁺ cells during flow cytometry. Dead cells can be eliminated using the viability dye 7-AAD. Flow cytometry and cell sorting can be performed on a FACSVantage (Becton Dickinson, San Jose, Calif. USA). Data files can be analyzed using Cell Quest software (Becton Dickinson).

We found that breast cancer cells were heterogeneous with respect to expression of a variety of cell surface-markers including CD44, CD24, and B38.1.

To determine whether these markers could distinguish tumorigenic from non-tumorigenic cells, flow cytometry was used to isolate cells that were positive or negative for each marker from first passage T1 or T2 cells. Cells were isolated by flow cytometry as described in FIG. 1, based upon expression of the indicated marker and assayed for the ability to form tumors after injection into the mammary fat pads of NOD/SCID mice. For twelve weeks, mice were examined weekly for tumors by observation and palpation. Then, all mice were necropsied to look for growths at injection sites that were too small to palpate. A “palpable tumor” is known to those in the medical arts as a tumor that is capable of being handled, touched, or felt. All tumors were readily apparent by visual inspection and palpation except for tumors from the CD24⁺ population which were only detected upon necropsy.

When 200,000-800,000 cells of each population were injected, all injections of CD44⁺ cells (8/8), B38.1³⁰ cells (8/8), or CD24^{-/low} cells (12/12) gave rise to visible tumors within twelve weeks of injection, but none of the CD44⁻ cell (0/8), or B38.1⁻ cell (0/8) injections formed detectable tumors (TABLE 2). The ratio of the number of tumors that formed/the number of injections that were performed is indicated for each population.

TABLE 2
Tumorigenicity of Different
Populations of Solid Tumor Stem Cells

| | # tumors/# of injections | | |
|---|---|---|---|
| Cells/injection | 8 × 10⁵ | 5 × 10⁵ | 2 × 10⁵ |
| T1 cells | | | |
| CD44⁻ | 0/2 | 0/2 | — |
| CD44⁺ | 2/2 | 2/2 | — |
| B38.1⁻ | 0/2 | 0/2 | — |
| B38.1⁺ | 2/2 | 2/2 | — |
| CD24⁺ | — | — | 1/6 |
| CD24⁻ | — | — | 6/6 |
| T2 cells | | | |
| CD44⁻ | 0/2 | 0/2 | — |
| CD44⁺ | 2/2 | 2/2 | — |
| B38.1⁻ | 0/2 | 0/2 | — |
| B38.1⁺ | 2/2 | 2/2 | — |
| CD24⁺ | — | — | 1/6 |
| CD24⁻ | — | — | 6/6 |

Although no tumors could be detected by palpation in the locations injected with CD24⁺ cells, two of twelve mice injected with CD24⁺ cells did contain small growths at the injection site that were detected upon necropsy. These growths most likely arose from the 1-3% of CD24⁻ cells that invariably contaminated the sorted CD24⁺ cells, or alternatively from CD24⁺ cells with reduced proliferative capacity (TABLE 2). Because the CD44⁺ cells were exclusively B38.1⁺, we focused on the CD44 and CD24 markers in subsequent assays.

Several antigens associated with normal cell types (LINEAGE markers CD2, CD3, CD10, CD16, CD18, CD31, CD64, and CD140b) were found not to be expressed by the cancer cells based on analyses of tumors that had been passaged multiple times in mice. By eliminating LINEAGE⁺ cells from unpassaged or early passage tumor cells, normal human leukocytes, endothelial cells, mesothelial cells and fibroblasts were eliminated. By microscopic examination, the LINEAGE⁻ tumor cells consistently had the appearance of neoplastic cells (FIG. 1g and FIG. 1h).

An average of 15% (range from 8% to 26%) of the LINEAGE⁻ cancer cells in tumors or pleural effusions were CD44⁺CD24^{-/low} (FIG. 1ato FIG. 1f). CD44⁺CD24^{-/low}LINEAGE⁻ cells or other populations of LINEAGE⁻ cancer cells that had been isolated from nine patients were injected into the breasts of mice (TABLE 3). When injecting unfractionated passaged T1or T2 cells, 50,000 cells consistently gave rise to tumors, but 10,000 cells gave rise to tumors in only a minority of cases (TABLE 3). In contrast, as few as 1,000 T1 or T2 CD44³⁰ CD24^{-/low}LINEAGE⁻ cells gave rise to tumors in all cases (TABLE 3). For T1 and T2, up to 20,000 cells that were CD44⁺ LINEAGE⁻ but CD24⁺ failed to form tumors (FIG. 1k). These data suggest that the CD44⁺CD24^{-/low} LINEAGE⁻ population is 10-50 fold enriched for the ability to form tumors in NOD/SCID mice relative to unfractionated tumor cells.

Whether the CD44⁺CD24^{-/low} LINEAGE⁻ cells were isolated from passaged tumors (T1, T2, T3) or from unpassaged tumors (T1, T4-T6, T8, T9), the cells were enriched for tumorigenic activity (TABLE 3). Note that T7 was the only one of nine cancers that we tested that did not fit this pattern (TABLE 3; see, below). CD44⁺CD24^{-/low}LINEAGE⁻ and CD24⁺LINEAGE⁻ cancer cells were consistently depleted of tumorigenic activity in both passaged and unpassaged samples (TABLE 3). Therefore, the xenograft and unpassaged patient tumors were composed of similar populations of phenotypically diverse cell types, and in both cases only the CD44+CD24 ^{-/low}LINEAGE⁻ cells had the capacity to proliferate to form new tumors (p<0.001).

TABLE 3 shows that tumorigenic breast cancer cells were highly enriched in the ESA⁺CD44⁺CD24^{-/low} population. Cells were isolated from first passage (designated Passage 1) Tumor 1, Tumor 2 and Tumor 3, second passage Tumor 3 (designated Passage 2), unpassaged T1, T4, T5, T6, T8 and T9 (designated Unpassaged), or unpassaged T7 cells (designated unpassaged T7). The indicated number of cells of each phenotype was injected into the breast of NOD/SCID mice.

TABLE 3
Tumorigenicity of Different Populations of Solid Tumor Stem Cells

| | 500,000 | 100,000 | 50,000 | 20,000 | 10,000 | 5,000 | 1,000 | 500 | 200 | 100 |
|---|---|---|---|---|---|---|---|---|---|---|
| Passage 1 | | | | | | | | | | |
| Unsorted | 8/8 | 8/8 | 10/10 | | 3/12 | | 0/12 | | | |
| CD44⁺CD24⁺ | | | | 0/10 | 0/10 | 0/14 | 0/10 | | | |
| CD44⁺CD24^{-/low} | | | | 10/10 | 10/10 | 14/14 | 10/10 | | | |
| CD44⁺CD24^{-/low}ESA⁺ | | | | | | | 10/10* | 4/4 | 4/4 | 1/6 |
| CD44⁺CD24^{-/low}ESA⁻ | | | | | | | 0/10* | 0/4 | 0/4 | 0/6 |
| Passage 2 | | | | | | | | | | |
| CD44⁺CD24⁺ | | | | | 0/9 | | | | | |
| CD44⁺CD24^{-/low} | | | | | 9/9 | | | | | |
| Unpassaged | | | | | | | | | | |
| CD44⁺CD24⁺ | | 0/3 | 0/4 | 0/8 | 1/13 | 0/2 | | | | |
| CD44⁺CD24^{-/low} | | 3/3 | 4/4 | | 11/13 | 1/1 | | | | |
| CD44⁺CD24^{-/low}ESA⁺ | | | | | | 2/2 | 2/2 | | | |
| CD44⁺CD24^{-/low}ESA⁻ | | | | | | 2/2^{#} | 0/2 | | | |
| Unpassaged T7 | | | | | | | | | | |
| CD44⁺CD24^{-/low} | | | | | 2/2 | | | | | |
| CD44⁺CD24⁺ | | | | | 2/2 | | | | | |
| CD44⁻CD24⁺ | | | | | 0/2 | | | | | |
| MCF-7 cells | | | | | 10/10 | | 10/10 | | | 0/20 |

#Tumor formation by T5 ESA⁻CD44⁺CD24^{-/low}LINEAGE⁻ cells was delayed by 2-4 weeks.
*2,000 cells were injected in these experiments. In addition to the markers that are shown, all sorted cells in all experiments were LINEAGE⁻, and the tumorigenic cells from T1, T2, and T3 were further selected as B38.1⁺.

The frequency of tumorigenic cells calculated by the modified maximum likelihood analysis method is ˜5/10⁵, according to the methods of Porter E H & Berry R J, Br. J. Cancer 17: 583 (1964) and Taswell C, .J Imnmunol. 126: 1614 (1981), if single tumorigenic cells were capable of forming tumors, and every transplanted tumorigenic cell gave rise to a tumor. Therefore, this calculation may underestimate the frequency of the tumorigenic cells (i.e., solid tumor stem cells), since the calculation does not take into account cell-cell interactions and local environment factors that may influence engraftmnent. CD44⁺CD24^{+/low} LINEAGE⁻ populations and CD44⁺CD24^{-/low}LINEAGE⁻ cells were isolated by flow cytometry as described in FIG. 1.

Limiting dilution analysis of MCF-7 cells showed that the proportion of clonogenic unsorted cells from this cell line was similar to that of sorted, enriched breast cancer stem cells from tumors. The mice were observed weekly for 4-6½ months or until the mice became sick from the tumors.

Twelve weeks after injection, the injection sites of 20, 000 tumorigenic CD44⁺CD24^{-/low}LINEAGE⁻ cells and 20,000 non-tumorigenic CD44⁺CD24^{+/low}LINEAGE⁻ cells were examined by histology. The CD44⁺CD24^{-/low}LINEAGE⁻ injection sites contained tumors approximately 1 cm in diameter while the CD44⁺CD24⁺LINEAGE⁻ injection sites contained no detectable tumors. Only normal mouse mammary tissue was seen by histology at the sites of the CD44⁺CD24⁺LINEAGE⁻ injections (FIG. 1i), whereas the tumors formed by CD44⁺CD24^{-/low}LINEAGE⁻ cells contained malignant cells as judged by hematoxylin and eosin stained sections (FIG. 1j). Even when CD44⁺CD24⁺LINEAGE⁻ injection sites from fifty-eight mice, each administered 1,000-50,000 cells, were examined after 16-29 weeks, no tumors were detected. Both the tumorigenic and non-tumorigenic subsets of LINEAGE⁻ cells from passaged and unpassaged tumors contained >95% cancer cells as judged by Wright staining or Papanicolaou staining and microscopic analysis (FIG. 1g and FIG. 1h).

In three of the tumors, further enrichment of tumorigenic activity was possible by isolating the ESA⁺ subset of the CD44³⁰CD24^{-/low} population. ESA (Epithelial Specific Antigen, Ep-CAM) expression distinguishes epithelial cancer cells from benign reactive mesothelial cells. Packeisen J et al., Hybridoma 18: 37-40, 1999). The CD44⁺CD24^{-/low}LINEAGE⁻ tumorigenic population typically accounted for approximately 8-25% of viable breast cancer cells, but the data suggest that in some tumors an even smaller population of tumorigenic cells may be identified by selecting the ESA subset.

When ESA⁺CD44⁺CD24^{-/low}LINEAGE⁻ cells were isolated from passaged T1, as few as 200 cells consistently formed tumors of approximately 1 cm 6 months after injection whereas 2000 ESA⁻CD44⁺CD24 ^{-/low}LINEAGE⁻ cells or 20,000 CD44⁺CD24⁺ cells always failed to form tumors (TABLE 1). These data suggest that the ESA⁺CD44⁺CD24^{-/low}LINEAGE⁻ population was more than 50 fold enriched for the ability to form tumors relative to unfractionated tumor cells (TABLE 1). The ESA⁺CD44⁺CD24^{-/low}LINEAGE⁻ population accounted for 2-4% of first passage T1cells (2.5-5% of cancer cells). The ESA⁺CD44⁺CD24^{-/low}LINEAGE⁻ population (0.6% of cancer cells) from unpassaged T5 cells was also enriched for tumorigenic activity compared to ESA⁻CD44⁺CD24^{-/low}LINEAGE⁻ cells, but both the ESA⁺ and ESA⁻ fractions had some tumorigenic activity (TABLE 1). Among unpassaged T5 cells, as few as 1000 ESA⁺CD44⁺CD24^{-/low}LINEAGE⁻ cells consistently formed tumors.

In a comedo subtype breast ductal adenocarcinoma that we designated T7, tumorigenic activity was observed in both the CD44⁺CD24^{-/low} and the CD44⁺CD24⁺ populations (TABLE 1, FIG. 1f). This suggests that the tumorigenic cells from some patients may differ in cell surface marker expression.

Phenotypic diversity in tumors arisingfrom solid tumor stem cells. The ability of small numbers of CD44⁺CD24^{-/low}LINEAGE⁻ tumorigenic cells to give rise to new tumors was reminiscent of the organogenic capacity of normal stem cells. Normal stem cells self-renew and give rise to phenotypically diverse cells with reduced proliferative potential. To test whether tumorigenic breast cancer cells also exhibit these properties, tumors arising from 200 ESA⁺CD44⁺CD24^{-/low}LINEAGE⁻ T1or 1,000 CD44^{+CD}24^{-/low}LINEAGE⁻ T2 cells were dissociated and analyzed by flow cytometry. The heterogeneous expression patterns of ESA, CD44 or CD24 in the secondary tumors resembled the phenotypic complexity of the original tumors from which the tumorigenic cells were derived (compare FIG. 2a and FIG. 2b with FIG. 2e and FIG. 2f). Within these secondary tumors, the CD44⁺CD24^{-/low}LINEAGE⁻ cells remained tumorigenic, while other populations of LINEAGE⁻ cancer cells remained non-tumorigenic Passage 2; TABLE 1). Thus tumorigenic cells gave rise to both additional CD44⁺CD24^{-/low}LINEAGE⁻ tumorigenic cells as well as to phenotypically diverse non-tumorigenic cells that recapitulated the complexity of the primary tumors from which the tumorigenic cells had been derived. These CD44⁺CD24^{-/low}LINEAGE⁻ tumorigenic cells from T1, T2 and T3 have now been serially passaged through four rounds of tumor formation in mice, yielding similar results in each round with no evidence of decreased proliferation. These results suggest that CD44⁺CD24^{-/low}LINEAGE⁻ tumorigenic cancer cells undergo processes analogous to the self-renewal and differentiation of normal stem cells.

Comparison of the cell cycle status of tumorigenic and non-tumorigenic cancer cells from T1revealed that both exhibited a similar cell cycle distribution (FIG. 2g and FIG. 2h). Therefore, neither population was enriched for cells at a particular stage of the cell-cycle, and the non-tumorigenic cells were able to undergo at least a limited number of divisions in the xenograft model.

The implications of prospectively identifying tumorigenic cancer cells. The tumorigenic CD44⁺CD24^{-/low}LINEAGE⁻ population shares with normal stem cells the ability to proliferate extensively, and to give rise to diverse cell types with reduced developmental or proliferative potential. The extensive proliferative potential of the tumorigenic population was demonstrated by the ability of as few as 200 passaged or 1000 unpassaged ESA⁺CD44⁺CD24^{-/low}LINEAGE⁻ cells to give rise to tumors (greater than 1 cm in diameter) that could be serially transplanted in NOD/SCID mice. The tumorigenic population from T1, T2 and T3 has now been isolated and serially passaged four times through NOD/SCID mice. This extensive proliferative potential contrasts with the bulk of CD44⁻ and/or CD24⁺cancer cells that lacked the ability to form detectable tumors. Not only was the CD44⁺CD24^{-/low}LINEAGE⁻ population of cells able to give rise to additional tumorigenic CD44⁺CD24^{-/low}LINEAGE⁻ cells, but they were also able to give rise to phenotypically diverse non-tumorigenic cells that composed the bulk of the tumors. This remained true even after two rounds of serial passaging. Thus, CD44⁺CD24^{-/low}LINEAGE⁻ cells from most tumors appear to exhibit properties of solid tumor stem cells.

Our data demonstrate there is a hierarchy of solid tumor cells in which only a fraction of the cells have the ability to proliferate extensively while other cells have only a limited proliferative potential. These results demonstrate that phenotypically distinct populations of solid tumor cells have an intrinsically greater capacity to proliferate extensively and form new tumors than other populations. In most tumors we could predict whether cancer cells were tumorigenic or depleted or tumorigenic activity based on marker expression. Although tumorigenic breast cancer cells were orders of magnitude more likely to form tumors than non-tumorigenic breast cancer cells, there may also be a stochastic component to tumorigenicity in the sense that not every injected tumorigenic cell formed a tumor. Breast cancer cell divisions are genetically unstable and individual breast cancer cells from the tumorigenic population may sometimes be unable to proliferate as a consequence of chromosomal aberrations acquired during mitosis. Murphy K L et al., FASEB Journal 14: 2291-2302 (2000).

The observation that eight of nine independent tumors contained a small population of tumorigenic cells with a common cell surface phenotype has profound implications for understanding solid tumor biology and the development of effective cancer therapies. The inability of current cancer treatments to cure metastatic disease may be due to ineffective killing of tumorigenic cells. If the non-tumorigenic cells are preferentially killed by particular therapies, then tumors may shrink but the remaining tumorigenic cells will drive tumor recurrence. By focusing on the tumorigenic population, one can identify critical proteins that are expressed by virtually all of the tumorigenic cells in a particular tumor. The prospective identification of the tumorigenic cancer cells should allow the identification of more effective therapeutic targets, diagnostic markers that detect the dissemination of tumorigenic cells, and more effective prognostic markers, by focusing on the tumorigenic cells rather than on more functionally heterogeneous collections of cancer cells.

Notch4 as a therapeutic target. We looked for the expression of proteins that may modulate key biological functions of the tumorigenic cells. Activation of the Notch receptor has previously been implicated in breast cancer and Notch signaling plays a role in transformation of cells transfected with an activated Ras oncogene. Berry L W et al., Development 124(4):925-36 (1997); Morrison S J et al., Cell 101(5): 499-510 (2000). Given the analogous properties of tumorigenic cancer cells and normal stem cells, we focussed on targets such as the Notch signaling pathway that are known to regulate the self-renewal of a variety of normal stem cells and the proliferation of cancer cell lines.

We have discovered that Notch4 plays a role both in tumorigenesis. Within an individual solid tumor, only a small subpopulation of tumorigenic cells expresses high levels of Notch4. An antibody that recognizes Notch4 blocks the growth of breast cancer tumor cells in vitro and in vivo. In one embodiment, the antibody binds to the extracellular domain of Notch4. In a particular embodiment, the antibody binds to the polypeptide region LLCVSVVRPRGLLCGSFPE (LeuLeuCysValSerValValArgProArgGlyLeuLeuCysGlySerPheProGlu) (SEQ ID NO:1). However, any anti-Notch4 antibody that inhibits Notch activation can be used to impair tumor survival.

We found by RT-PCR that T1 CD44⁺CD24^{-/low}LINEAGE⁻ tumorigenic cells expressed Notch4 (FIG. 3a). We therefore tested the effect of Notch activation in breast cancer cells by exposing the cells in culture to a soluble form of the Notch ligand Delta, Delta-Fc. Morrison S J et al., Cell 101: 499-510 (2000). We found that soluble Delta increased the number of colonies formed by unfractionated T1cancer cells in culture five-fold (FIG. 3b). Thus, Notch activation promoted the survival or proliferation of clonogenic cancer cells, i.e., solid tumor stem cells.

To test whether inhibition of Notch4 signaling would reduce survival or proliferation, we exposed the cells to a polyclonal, blocking antibody against Notch4 that reduced Notch pathway reporter gene activation (FIG. 3c). The anti-Notch4 antibody which was purchased from Santa Cruz Products (Santa Cruz, Calif. USA). The antibody binds to the polypeptide region LLCVSVVRPRGLLCGSFPE (LeuLeuCysValSerValValArgProArgGlyLeuLeuCysGlySerPheProGlu) (SEQ ID NO:1). For the Notch reporter assay, the HES-1—Luciferase reporter construct was made as described by Liu A Y et al., Proc. Natl. Acad. Sci. USA 94: 10705-10710 (1997). The fragment of the HES-1 murine gene between −194 and +160 was amplified by PCR and subdloned into a pGL2 basic vector (Promega) between the KpnI and Bgl II sites. MCF-7 cells were co-transfected with the HES-1-luc construct and pSV2Neo and selected in medium containing geneticin.

For RT-PCR, RNA was isolated using Trizol (Gibco BRL). For the Notch4 gene expression analysis, reverse transcription of 0.2 mg RNA isolated from T1, MCF-7 and MCF-10A cells , was done using a gene specific anchor primer 5′-TCCTCCTGCTCCTACTCCCGAGA-3′ (SEQ ID NO: 2). The Notch4 fragment was amplified using the following primers: 5′-TGAGCCCTGGGAACCCTCGCTGGATGGA-3′ (SEQ ID NO: 3) and 5′-AGCCCCTTCCAGCAGCGTCAGCAGAT-3′ (SEQ ID NO: 4).

The transfected MCF-7 cells were cocultivated in 12-well plates in the presence and absence of the Notch4 polyclonal antibody (Santa Cruz; 20 μg/ml final concentration), soluble Delta-Fc (Morrison S J et al., Cell 101: 499-510 (2000)) or the Notch4 antibody blocking peptide (4 mg/100 ml final concentration, Santa Cruz Products), LLCVSVVRPRGLLCGSFPE (LeuLeuCysValSerValValArgProArgGlyLeuLeuCysGlySerPheProGlu) (SEQ ID NO:1).

Luciferase assays were performed as described by Jarriault S et al., Nature 377: 355-358 (1995). Delta-Fc or Fc control proteins were concentrated from the supernatant of 293 cells that were engineered to secrete them according to the methods of Morrison S J et al., Cell 101: 499-510 (2000). Delta-Fc or Fc control proteins were added to breast cancer cell cultures along with a cross-linking anti-Fc antibody (Jackson Imunoresearch) as previously described by Morrison S J et al., Cell 101: 499-510 (2000).

When cells were exposed to this anti-Notch4 antibody, colony formation was almost completely inhibited (FIG. 3b). This inhibition was nearly completely eliminated by pre-incubation of the antibody with the Notch4 peptide against which the antibody was generated, which presumably prevented binding of the antibody to Notch4 on the cell surface (FIG. 3b). The anti-Notch4 antibody also inhibited colony formation by the MCF-7 breast cancer cell line, but not the MCF-10 cell line (Soule H D et al., Cancer Research 50, 6075-6086 (1990)) that was derived from normal breast epithelium. To determine whether the anti-Notch4 antibody inhibited tumor formation, 100-500 ESA³⁰ CD44⁺CD24^{-/low}LINEAGE⁻ cells incubated with either control buffer or the anti-Notch4 antibody were injected into mice. nine of eleven injections of 200-500 untreated cells and one of eleven injections of 100 untreated cells formed tumors (FIG. 3d). By contrast, injection of 100 or 200 cells treated with anti-Notch4 antibody failed to form tumors and tumor formation by 500 antibody-treated cells was delayed relative to control cells (FIG. 3d).

Notch4 signaling provides a survival signal to tumor-initiating cells. We next studied the mechanism by which anti-Notch4 antibody inhibited colony formation. Notch stimulation has been shown to promote self-renewal in some circumstances, inhibit proliferation in other circumstances, and to promote survival in other cases. To distinguish between these possibilities, unfractionated cancer cells isolated from four tumors, MCF-7 cells and MCF-10 cells were analyzed for proliferation and cell death after exposure to the anti-Notch4 antibody. There was no significant difference in the cell cycle distribution of MCF-7 cells (which expressed Notch4, supplemental FIG. 1), exposed to the anti-Notch4 antibody when compared to untreated cells twenty-four hours after antibody exposure (FIG. 4a). However, exposure of MCF-7 cells, unfractionated tumor cells isolated from T10, or T1 ESA⁺CD44⁺CD24^{-/low}LINEAGE⁻ breast cancer tumorigenic cells, but not MCF10 cells or unfractionated tumor cells isolated from T7 and T8, to the anti-Notch4 blocking antibody led to the accumulation of cells with degraded DNA characteristic of apoptosis and to the activation of caspase 3/7 in a significant percentage of the cells thirty-six hours after antibody exposure (FIG. 4b and FIG. 4c).

For the apoptosis assays, tumorigenic T1 cells (ESA⁺CD44⁺CD24^{-/low}LINEAGE⁻) or LINEAGE⁻ tumor cells from T7, T8 and T10 were sorted by flow cytometry and grown on collagen coated tissue culture plates. The T10 tumorigenic cells have not yet been characterized. Anti-Notch4 polyclonal antibody (Santa Cruz , Calif. USA) was then added to the medium (20 mg/ml final concentration) while PBS was added to the control plates. To block the anti-Notch4 antibody, the anti-Notch4 antibody was pre-incubated with the blocking peptide (Santa Cruz, Calif. USA) on ice for 30 minutes after which it was added to the medium. After 48 hrs, cells were trypsinized and collected. 10⁵ cells were suspended in HBSS 2% heat inactivated calf serum and then assayed for apoptosis using FAM-DEVD-FMK, a caroxyfluorescein labeled peptide substrate specific to caspases 3 and 7 (CaspaTag™ Caspase Activity Kit, Intergen Company, New York USA) to detect active caspases in living cells. Caspase positive cells were distinguished from the negative ones using FACSVantage flow cytometer (Becton Dickinson, California USA). PI staining for cell cycle and apoptosis was performed as described by Clarke M F et al., Proc. Natl. Acad. Sci. USA 92:11024-11028, (1995).

These data suggest that, in some de novo human tumors, Notch pathway activation provides a necessary survival signal to the tumorigenic population of breast cancer cells.

Maniac Fringe as a therapeutic target for breast cancer stem cells. Proteins with knife-edge names such as Jagged (Shimizu et al., Journal of Biological Chemistry 274(46) 32961-9 (1999); Jarriault et al., Molecular and Cellular Biology 18: 7423-7431 (1998)), Serrate, and Delta (and variants of each, such as Delta1, Delta2, Delta3, Delta4, Jagged 1 and Jagged2, LAG-2 and APX-1 in C. elegans), bind to the Notch receptor and activate a downstream signaling pathway that prevents neighboring cells from becoming neural progenitors. The recently identified ligand is D114 is a Notch ligand of the Delta family expressed in arterial endothelium. Shutter et al., Genes Dev 14(11): 1313-8 (2000)).

Notch ligands may bind and activate Notch family receptors promiscuously. The expression of other genes, like Fringe family members (Panin et al, Nature 387(6636): 908-912 (1997)), may modify the interactions of Notch receptors with Notch ligands. Numb family members may also modify Notch signaling intracellularly.

Ligand binding to Notch results in activation of a presenilin-1-dependent gamma-secretase-like protein that cleaves Notch. De Strooper et al., Nature 398: 518-522 (1999), Mumm et al., Molecular Cell. 5: 197-206 (2000). Cleavage in the extracellular region may involve a furin-like convertase. Logeat et al., Proceedings of the National Academy of Sciences of the USA 95: 8108-8112 (1998). The intracellular domain is released and transactivates genes by associating with the DNA binding protein RBP-J. Kato et al., Development 124: 4133-4141 (1997)). Notch1, Notch2 and Notch4 are thought to transactivate genes such as members of the Enhancer of Split (HES) family, while Notch3 signaling may be inhibitory. Beatus et al., Development 126: 3925-3935 (1999). Finally, secreted proteins in the Fringe family bind to the Notch receptors and modify their function. Zhang & Gridley, Nature 394 (1998).

Inhibitors of Notch signaling (such as Numb and Numb-like; or antibodies or small molecules that block Notch activation) can be used in the methods of the invention to inhibit solid tumor stem cells. In this manner, the Notch pathway is modified to kill or inhibit the proliferation of solid tumor stem cells.

Since the Notch signaling pathway appears to play a critical role in the proliferation of T1 cancer stem cells and MCF-7 cells, we determined the expression of Notch4 and members of the Fringe family by different populations of Tumor 1 cancer cells. Flow cytometry showed that both the tumorigenic and non-tumorigenic cancer cells expressed Notch4. We next examined two tumors for expression of members of the Fringe family. The three Fringe proteins, Manic, Lunatic and Radical, all glycosylate Notch receptors and modulate receptor signaling. However, the effect of a particular Fringe on signal transduction via each of the four Notch receptors can differ. Furthermore, each Fringe is thought to glycosylate a particular Notch receptor at different sites, resulting in a differential response to a particular ligand.

RNA was isolated from solid tumor cells using Trizol (Gibco BRL, Rockfill, Md.). After reverse transcription, the EGF-R and the Her2/neu fragments were amplified using the following primers: EGF-R, 5′-GCCAGGAATTGAGAGTCTCA-3′ (SEQ ID NO:5), 5′-AAGCCTGTTATTCTGCCTTTTA-3′ (SEQ ID NO:6), 5′-CCACCAATCCAACATCCAGA-3′ (SEQ ID NO:7) and 5′-AACGCCTGTCATAGAGTAG-3′ (SEQ ID NO:8); Her2/neu, 5′-CACAGGTTACCTATACATCT-3′ (SEQ ID NO:9), 5′-GGACAGCCTGCCTGACCTCA-3′ (SEQ ID NO:10), 5′-CCACAGGGAGTATGTGAATG-3′ (SEQ ID NO:11), and 5′-TTTGCCGTGGGACCCTGAGT-3′ (SEQ ID NO:12) respectively. The RT-PCR for the Fringe transcripts were done using the following external primers, for Manic fringe, 5′-GGCTGAATTGAAAAAGGGCAG-3′ (SEQ ID NO:13) and 5′-AGCAGGAAGATGGGGAGTGG-3′ (SEQ ID NO:14), for Radical Fringe, 5′-CCGAGAGGGTCCAGGGTGGC-3′ (SEQ ID NO:15)and 5′-CCTGAGGGAGCCCACTGAGC-3′ (SEQ ID NO:16), and for Lunatic Fringe 5′-CCAGCCTGGACAGGCCCATC-3′ (SEQ ID NO:17), and 5′-ACGGCCTGCCTGGCTTGGAG-3′ (SEQ ID NO:18) respectively and the following internal primers.

RT-PCR using 0.1 ug of unseparated tumor RNA demonstrated that T1 cells expressed Manic Fringe, Radical Fringe and Lunatic Fringe whereas RT-PCR of 100 ESA⁺B38.1⁺CD24^{-/lo}LINEAGE⁻ (tumorigenic) cells demonstrated that these cells expressed Manic Fringe, but not Lunatic Fringe or Radical Fringe. When examined by single cell RT-PCR, all six T1tumorigenic cells expressed Manic Fringe, but only two of six non-tumorigenic cells did so. By contrast, all of the non-tumorigenic, but none of the tumorigenic, single cells examined expressed Lunatic Fringe and Radical Fringe. Fringe expression by unpassaged T5 stem cells and non-tumorigenic cells was determined to see if there was a difference in expression by the different populations of unpassaged breast cancer cells. Single cell RT-PCR showed that all six of the T5 breast cancer stem cells tested expressed Manic Fringe, but only ⅙ of the cells expressed Lunatic Fringe and only one of six cells tested expressed Radical Fringe respectively. By contrast, all of the non-tumorigenic cells tested expressed Lunatic Fringe and five of six expressed Radical Fringe, but only one of six cells expressed Manic Fringe. Thus, the expression of the different Fringe genes by the tumorigenic and non-tumorigenic unpassaged T5 cells reflected the pattern seen in the passaged T1 cells. Manic Fringe has been implicated in oncogenic transformation. These data demonstrate the differential expression by tumorigenic and non-tumorigenic neoplastic cells of genes involved in a biologically relevant pathway that appears to regulate tumorigenesis in these cells. Whether the different Fringe genes play a direct role in breast cancer cell fate decisions or their differential expression is simply associated with a particular cell population remains to be tested.

Selective targeting of solid tumor stein cells. We determined the expression of EGF-R, Her2/neu, Notch4, Manic Fringe, Lunatic Fringe and Radical Fringe by tumorigenic breast cancer cells (i.e., solid tumor stem cells, in particular Tumor 1 (T1) cells) EGF-R and HER2/neu are potential therapeutic targets that have been implicated in breast cancer cell proliferation.

Flow cytometry was used to isolate subpopulations of T1cells that had been passaged once in NOD/SCID mice. Cells were stained with anti-EGF-R, anti-CD24, anti-Lineage, anti-mouse-H2K, and 7AAD or anti-HER2/neu, anti-CD24, anti-Lineage, anti-mouse-H2K, and 7AAD. Dead cells (7AAD⁺), mouse cells (H2K⁺) and LINEAGE⁺ cells were eliminated from all analyses. RT-PCR using nested primers was also performed to detect EGF-R or to detect HER2/neu in one cell per sample in panels or ten cells per sample in panels.

By focusing on the tumorigenic population of cells in T1, we were able to identify Her2/neu Notch4 and Manic Fringe, while potentially eliminating EGF-R Radical Fringe and Lunatic Fringe, as possible therapeutic targets in this particular tumor. While most of the tumorigenic cells expressed detectable levels of HER2/neu protein and mRNA, we were not able to detect expression of EGF-R in most tumorigenic cells.

Tumorigenic T1 cells stained with lower levels of anti-EGF-R antibody than non-tumorigenic cells, and EGF-R expression could not be detected at the single cell level in tumorigenic cells. To test whether cells that did not express detectable levels of the EGF-R were tumorigenic, 1,000-2,000 tumorigenic cells were also sorted with respect to EGFR expression and injected into NOD/SCID mice. Tumors formed in four out of four cases, confirming that the EGF-R⁻ cells are tumorigenic. In contrast, we could not detect a substantial difference in HER2/neu expression between tumorigenic and non-tumorigenic T1 cells. As expected, 1,000-2,000 HER2/neu⁺ cells gave rise to tumors in four out of four cases. These observations suggest that there can be differences in the expression of therapeutic targets between the tumorigenic and non-tumorigenic populations.

Since therapies that kill only non-tumorigenic cancer cells may produce temporary tumor regression but will not be able to eradicate the tumor, these results suggest that agents that kill HER2/neu expressing cells might be more effective than those that kill EGF-R expressing cells in this tumor. Other breast cancer tumors may manifest different patterns of expression of these genes. Thus, theprospective identification and isolation of tumorigenic cells should allow a more focused biological, biochemical and molecular characterization of the factors critical for tumor formation and permit the specific targeting of therapeutic agents to this cell population, resulting in the development of more effective cancer treatments.

Solid stem cells and solid stem cell progeny of the invention can be used in methods of determining the effect of a biological agents on solid tumor cells, e.g., for diagnosis, treatment or a combination of diagnosis and treatment. The term “agent” or “compound” refers to any agent (including a virus, protein, peptide, amino acid, lipid, carbohydrate, nucleic acid, nucleotide, drug, antibody, prodrug, other “biomolecule” or other substance) that may have an effect on tumor cells whether such effect is harmful, beneficial, or otherwise. The ability of various biological agents to increase, decrease, or modify in some other way the number and nature of the solid tumor stem cells and solid tumor stem cell progeny can be assayed by methods known to those of skill in the drug discovery art.

In one embodiment, a pharmaceutical composition containing a Notch4 ligand, an anti-Notch4 antibody, or other therapeutic agent that acts as an agonist or antagonist of proteins in the Notch signal transduction/response pathway can be administered by any effective method. For example, a physiologically appropriate solution containing an effective concentration of anti-Notch therapeutic agent can be administered topically, intraocularly, parenterally, orally, intranasally, intravenously, intramuscularly, subcutaneously or by any other effective means. In particular, the anti-Notch therapeutic agent may be directly injected into a target cancer or tumor tissue by a needle in amounts effective to treat the tumor cells of the target tissue. Alternatively, a solid tumor present in a body cavity such as in the eye, gastrointestinal tract, genitourinary tract (e.g., the urinary bladder), pulmonary and bronchial system and the like can receive a physiologically appropriate composition (e.g., a solution such as a saline or phosphate buffer, a suspension, or an emulsion, which is sterile) containing an effective concentration of anti-Notch4 therapeutic agent via direct injection with a needle or via a catheter or other delivery tube placed into the cancer or tumor afflicted hollow organ. Any effective imaging device such as X-ray, sonogram, or fiber-optic visualization system may be used to locate the target tissue and guide the needle or catheter tube. In another alternative, a physiologically appropriate solution containing an effective concentration of anti-Notch therapeutic agent can be administered systemically into the blood circulation to treat a cancer or tumor that cannot be directly reached or anatomically isolated. All such manipulations have in common the goal of placing the anti-Notch4 agent in sufficient contact with the target tumor to permit the anti-Notch4 agent to contact, transduce or transfect the tumor cells (depending on the nature of the agent).

In treating a cancer patient who has a solid tumor, a therapeutically effective amount of an anti-Notch therapeutic agent can be administered. A “therapeutically effective” dose refers to that amount of the compound sufficient to result in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ as determined in cell culture. Such information can be used to more accurately determine useful doses in humans. Levels in plasma may be measured, for example, by high performance liquid chromatography (HPLC).

In another embodiment, a biomolecule or biological agent selectively targeted to a solid tumor stem cell can use gene therapy strategies. For example, the biomolecule can be a gene therapy suicide vector targeted to solid tumor stem cells using markers expressed by the solid tumor stem cells. In one embodiment, the vector is an adenoviral vector which has been redirected to bind to the B38.1 marker. We conjugated anti-fiber and the B38.1 antibodies with the Prolinx (Prolinx, Inc., Bothell, Wash., USA) method (see Douglas J T et al., Nature Biotechnology. 14(11):1574-8 (1996)). When we mixed the modified anti-knob and anti-B38.1 antibodies together, they became cross-linked and generated the bi-specific conjugate. The anti-fiber antibody part of the conjugate can bind to the adenovirus, while the anti-B38.1 moiety can bind to the breast cancer stem cell. Incubation of the AdLacZ virus with the anti-fiber alone blocks the infectivity of the virus. The infectivity of virus incubated with the bi-specific conjugate is restored only in the cells that express high levels of the B38.1 antigen. The re-targeting is specific, because it can be inhibited by free B38.1 antibody. The conclusion is that a bi-specific conjugate can modifies the infectivity of a vector, blocking its natural tropism and directing the infection to cells that express the solid tumor stem cell surface marker.

One skilled in the oncological art of can understand that the vector is to be administered in a composition comprising the vector together with a carrier or vehicle suitable for maintaining the transduction or transfection efficiency of the chosen vector and promoting a safe infusion. Such a carrier may be a pH balanced physiological buffer, such as a phosphate, citrate or bicarbonate buffers a saline solution, a slow release composition and any other substance useful for safely and effectively placing the targeted agent in contact with solid tumor stem cells to be treated.

Depending on the specific conditions being treated, agents may be formulated and administered systemically or locally. Techniques for formulation and administration may be found in Remington's Pharmaceutical Sciences, 20th ed. (Mack Publishing Co., Easton, Pa.). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents of the invention maybe formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

In addition to the active ingredients, these pharmaceutical compositions may contain suitable pharmaceutically acceptable carriers comprising excipients and auxiliaries, which facilitate processing of the active compounds into preparations which can be used pharmaceutically. The preparations formulated for oral administration may be in the form of tablets, capsules, or solutions. The pharmaceutical compositions of the present invention may be manufactured in a manner that is itself known, e.g., by means of conventional mixing, dissolving, granulating, levigating, emulsifying, encapsulating, entrapping or lyophilizing processes. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

The exact formulation, route of administration and dosage can be chosen by the individual physician in view of the patient's condition (see e.g. Fingl et al., In The Pharmacological Basis of Therapeutics, Ch. 1, pg. 1 (1975)). The attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the clinical disorder of interest can vary with the severity of the condition to be treated and the route of administration. See, Merck Index: An Encyclopedia of Chemicals, Drugs and Biologicals, 12 Edition (CRC Press 1996); Physicians'Desk Reference 55th Edition (2000)). The severity of the condition may, for example, be evaluated, in part, by appropriate prognostic evaluation methods. Further, the dose and perhaps dose frequency, also vary according to the age, body weight, and response of the individual patient. A program comparable to that discussed above may be used in veterinary medicine.

The details of one or more embodiments of the invention have been set forth in the accompanying description above. Although any methods and materials similar or equivalent to those described herein can be used in the practice or testing of the present invention, the preferred methods and materials are described. Other features, objects, and advantages of the invention will be apparent from the description and from the claims.

In the specification and the appended claims, the singular forms include plural referents. Unless defined otherwise in this specification, all technical and scientific terms used herein have the same meaning as commonly understood by one of ordinary skill in the art to which this invention belongs. All patents and publications cited in this specification are incorporated by reference.

1. A method for reducing the size of a solid tumor, comprising the step of:
contacting the cells of the solid tumor with a therapeutically effective amount of an agent directed against a Notch4 polypeptide.

2. The method of claim 1, wherein the therapeutically effective amount is an amount sufficient to cause cell death of or inhibit the proliferation of solid tumor stem cells in the solid tumor.

3. The method of claim 1, wherein the agent is an antibody, peptide or small molecule directed against a Notch4 polypeptide.

4. The method of claim 3, wherein the antibody, peptide or small molecule is directed against the extracellular domain of Notch4.

5. A method for reducing the size of a solid tumor, comprising:
contacting the cells of the solid tumor with a therapeutically effective amount of an agent that modulates the activity of a Notch4 ligand.

6. The method of claim 5, wherein the Notch4 ligand is selected from the group consisting of Delta 1, Delta 2, Delta-like ligand 4 (D114), Jagged 1 and Jagged 2.

7. The method of claim 5, wherein the agent is a Notch ligand agonist.

8. The method of claim 5, wherein the agent is a Notch ligand antagonist.

9. A method for reducing the size of a solid tumor, comprising:
contacting the cells of the solid tumor with a therapeutically effective amount of an agent that modulates the activity of Maniac Fringe.

10. The method of claim 9, wherein the agent is a Maniac Fringe agonist.

11. The method of claim 9, wherein the agent is a Maniac Fringe antagonist.

12. A method for killing or inhibiting the proliferation of solid tumor stem cells, comprising the step of:
contacting the cells of a solid tumor with an agent or combination of agents selectively targeted to the solid tumor stem cells of the solid tumor, wherein the agent or combination of agents kills or inhibits the proliferation of solid tumor stem cells.

13. The method of claim 12, further comprising the step of:
identifying the death of or the prevention of the growth of solid tumor stem cells in the solid tumor following contact by the agent or combination of agents.

14. The method of claim 12, wherein the killing is by the activation of cell death in the solid tumor stem cells.

15. The method of claim 14, wherein the cell death is apoptosis.

16. The method of claim 12, wherein the agent or combination of agents inhibits Notch4 signaling.

17. The method of claim 12, wherein the agent is an antibody, peptide or small molecule directed against a Notch4 polypeptide.

18. The method of claim 12, wherein the antibody, peptide or small molecule is directed against the extracellular domain of Notch4.

19. The method of claim 12, wherein the agent or combination of agents modulates the activity of a Notch4 ligand.

20. The method of claim 19, wherein the Notch4 ligand is selected from the group consisting of Delta 1, Delta 2, Delta-like ligand 4 (D114), Jagged 1 and Jagged 2.

21. The method of claim 12, wherein the agent or combination of agents modulates the activity of Maniac Fringe.

22. The method of claim 12, wherein the solid tumor stem cells express at least one marker selected from the group consisting of CD44, epithelial specific antigen (ESA), and B38.1.

23. The method of claim 12, wherein the solid tumor stem cells express the cell surface marker CD44.

24. The method of claim 12, wherein the solid tumor stem cells express the cell surface marker epithelial specific antigen (ESA).

25. The method of claim 12, wherein the solid tumor stem cells express the cell surface marker B38.1.

26. The method of claim 12, wherein the solid tumor stem cells express lower levels of the marker CD24 than the mean expression of CD24 by non-tumorigenic cancer cells of the solid tumor.

27. The method of claim 12, wherein the solid tumor stem cells fail to express at least one LINEAGE marker selected from the group consisting of CD2, CD3, CD10, CD14, CD16, CD31, CD45, CD64, and CD140b.

28. The method of claim 12, wherein the solid tumor is an epithelial cancer or a sarcoma

29. The method of claim 28, wherein the epithelial cancer is a breast cancer or an ovarian cancer.

30. A method for reducing the size of a solid tumor, comprising the step of:
contacting the cells of the solid tumor in vivo with an agent or combination of agents selectively targeted to the solid tumor stem cells of the solid tumor, wherein the agent or combination of agents kills or inhibits the proliferation of solid tumor stem cells.

31. The method of claim 30, further comprising the step of:
identifying the death of or the prevention of the growth of solid tumor stem cells in the solid tumor following contact by the agent or combination of agents.

32. The method of claim 30, wherein the killing is by the activation of cell death in the solid tumor stem cells.

33. The method of claim 32, wherein the cell death is apoptosis.

34. The method of claim 30, wherein the agent or combination of agents inhibits Notch-4 signaling.

35. The method of claim 30, wherein the agent is an antibody, peptide or small molecule directed against a Notch4 polypeptide.

36. The method of claim 35, wherein the antibody, peptide or small molecule is directed against the extracellular domain of Notch4.

37. The method of claim 30, wherein the agent or combination of agents modulates the activity of a Notch ligand.

38. The method of claim 30, wherein the Notch4 ligand is selected from the group consisting of Delta 1, Delta 2, Delta-like ligand 4 (D114), Jagged 1 and Jagged 2.

39. The method of claim 30, wherein the agent or combination of agents modulates the activity of Maniac Fringe.

40. The method of claim 30, wherein the solid tumor stem cells express at least one marker selected from the group consisting of CD44, epithelial specific antigen (ESA). and B38.1.

41. The method of claim 30, wherein the solid tumor stem cells express the cell surface marker CD44.

42. The method of claim 30, wherein the solid tumor stem cells express the cell surface marker epithelial specific antigen (ESA).

43. The method of claim 30, wherein the solid tumor stem cells express the cell surface marker B38.1.

44. The method of claim 30, wherein the solid tumor stem cells fail to express at least one LINEAGE marker selected from the group consisting of CD2, CD3, CD10, CD14, CD16, CD31, CD45, CD64, and CD140b.

45. The method of claim 30, wherein the solid tumor stem cells express lower levels of the marker CD24 than the mean expression of CD24 by non-tumorigenic cancer cells of the solid tumor.

46. The method of claim 30, wherein the solid tumor is an epithelial cancer or a sarcoma.

47. The method of claim 46, wherein the epithelial cancer is a breast cancer or an ovarian cancer.

48. A method for selectively targeting a solid tumor stem cell, comprising the steps of:
(a) identifying a marker present on a solid tumor stem cell;
(b) obtaining a biomolecule or set of biomolecules that selectively binds to the marker present on the solid tumor stem cell.

49. The method of claim 48, wherein the biomolecule genetically modifies the targeted solid tumor stem cell.

50. The method of claim 49, wherein the genetic modification results in solid tumor stem cell death.

51. The method of claim 48 wherein the biomolecule or set of biomolecules comprises a bi-specific conjugate.

52. The method of claim 48, wherein the biomolecule or set of biomolecules comprises an adenoviral vector.

53. The method of claim 49, wherein the adenoviral vector is selectively targeted to a solid tumor stem cell.

54. A biomolecule or set of biomolecules that is selectively targeted to solid tumor stem cell.

55. The method of claim 54, wherein the biomolecule genetically modifies the targeted solid tumor stem cell.

56. The method of claim 55, wherein the genetic modification results in solid tumor stem cell death.

57. The method of claim 54, wherein the biomolecule or set of biomolecules comprises a bi-specific conjugate.

58. The method of claim 54, wherein the biomolecule or set of biomolecules comprises an adenoviral vector.

59. The method of claim 58, wherein the adenoviral vector is selectively targeted to a solid tumor stem cell.

60. A method for forming a tumor in an animal, comprising:
introducing a cell dose of purified solid tumor stem cells into the animal, wherein:
(a) the solid tumor stem cell is derived from a solid tumor;
(b) the solid tumor stem cell population is enriched at least 2-fold relative to unfractionated tumor cells.

61. The method of claim 60, wherein the animal is an immunocompromised animal.

62. The method of claim 60, wherein the animal is a mammal.

63. The method of claim 62, wherein the mammal is an immunocompromised mammal.

64. The method of claim 62, wherein the mammal is a mouse.

65. The method of claim 64, wherein the mouse is an immunocompromised mouse.

66. The method of claim 65, wherein the immunocompromised mouse is selected from the group consisting of nude mouse, SCID mouse, NOD/SCID mouse, Beige/SCID mouse; and β2 microglobin deficient NOD/SCID mouse.

67. The method of claim 60, wherein the number of cells in the cell dose is between about 100 cells and about 5×10⁵ cells.

68. The method of claim 60, wherein the number of cells in the cell dose is about between about 100 cells and 500 cells.

69. The method of claim 60, wherein the number of cells in the cell dose is between about 100 cells and 200 cells.

70. The method of claim 60, wherein the number of cells in the cell dose is about 100 cells.

71. The method of claim 60, wherein the solid tumor stem cell expresses at least one marker selected from the group consisting of CD44, epithelial specific antigen (ESA), and B38.1.

72. The method of claim 60, wherein the solid tumor stem cell expresses the cell surface marker CD44.

73. The method of claim 60, wherein the solid tumor stem cell expresses the cell surface marker epithelial specific antigen (ESA).

74. The method of claim 60, wherein the solid tumor stem cell expresses the cell surface marker B38.1.

75. The method of claim 60, wherein the solid tumor stem cell expresses lower levels of the marker CD24 than the mean expression of CD24 by non-tumorigenic cancer cells derived from the solid tumor.

76. The method of claim 60, wherein the solid tumor stem cell does not express detectable levels of one or more LINEAGE markers, wherein a LINEAGE marker is selected from the group consisting of CD2, CD3, CD10, CD14, CD16, CD31, CD45, CD64, and CD140b.
